(19) United States
(12) Patent Application Publication
(10) Pub. No.: US 2026/0014018 A1
(43) Pub. Date: Jan. 15, 2026

(54) METHOD AND DEVICE FOR ALTERNATELY APPLYING HOT AND COLD TO AN APPLICATION SURFACE, NOTABLY THE EPIDERMIS OF A USER

(71) Applicant: L'OREAL, PARIS (FR)

(72) Inventors: Matthieu JACOB, CLICHY (FR); Karolina ABDEL BARY, CLICHY (FR)

(73) Assignee: L'OREAL, PARIS (FR)

(21) Appl. No.: 18/994,297

(22) PCT Filed: Jul. 18, 2023
(86) PCT No.: PCT/EP2023/069957

§ 371 Date: Jan. 14, 2025

(30) Foreign Application Priority Data
Jul. 28, 2022 (FR) 2207785

(51) Int. Cl. A61F 7/00; A61F 7/02

(52) U.S. Cl. CPC A61F 7/007; A61F 2007/0052; A61F 2007/0075; A61F 2007/0087; A61F 2007/0296; A61F 2007/0298

(57) ABSTRACT

Method (40) for alternately applying hot and cold to an application surface, notably the epidermis of a user, with the aid of a device for alternately applying hot and cold comprising a first end piece and a second end piece.

According to the method, a hot temperature of between 35° C. and 48° C. is applied to the application surface by the first end piece for a period of between 2 min and 6 min, the application device is pivoted by at most a quarter turn, for a period of less than or equal to 5 seconds, and a cold temperature of between 0° C. and 15° C. is applied to the application surface by the second end piece for a period of between 2 min and 6 min.

The present invention relates to the field of devices for cosmetic treatment of the epidermis of a user, notably the face.

The present invention also relates to the penetration into the skin of a cosmetic formulation or of a care formulation.

The term “cosmetic product” is understood, notably within the meaning of the present invention, to mean a product as defined in Regulation (EC) No 1223/2009 of the European Parliament and of the Council of 30 Nov. 2009 relating to cosmetic products.

More particularly, the present invention relates to a device and to a method configured to locally alternately obtain a feeling of hot or cold on the epidermis of a user.

Document FR 2 934 893-A1 (Cazaux et al.) is known which describes a cosmetic apparatus intended to produce cold for the purpose of cooling a cosmetic product and then, by application, cooling the skin of a user. The cosmetic apparatus comprises what is referred to as a “Peltier-effect” module, an aluminium transfer pad disposed on one of the faces of said module and a radiator-fan assembly attached to the other face of said module, opposite the transfer pad. The transfer pad has a convex shape and is intended to receive a cosmetic product, such as a cream, for the purpose of applying it to the epidermis of a user.

“Peltier effect” is understood to mean any physical phenomenon of movement of heat in the presence of an electrical current passing through conductive materials of different natures which are linked by junctions, one part of which absorbs heat while the other part releases heat. The Peltier effect is known and will not be described in any more detail.

However, the solution proposed in that document implies an application surface of significant size taking account of the dimensioning of the Peltier-effect module and therefore does not allow the cosmetic product to be applied precisely. Moreover, the significant dimensions of such an apparatus make it difficult to dissipate heat energy from the Peltier-effect module, and this may result in a diffusion of hot air towards the application area of the cosmetic product.

The application device described in document WO 2015/039990-A1 (L'Oréal) is also known which makes it possible to provide a feeling of cold with precision to narrow body areas by integrating a device for generating cold directly into the interior of said application device. However, the design and the dimensioning of such a device is particularly complex due to the integration of a device for generating cold.

Furthermore, such a device does not make it possible to alternate the application of cold and of hot to the user's epidermis.

There is thus a need to improve the devices and methods that are capable of alternating a feeling of cold or of hot on an application surface of a cosmetic product, notably the user's epidermis.

The subject of the invention is a method for alternately applying hot and cold to an application surface, notably the epidermis of a user, with the aid of a device for alternately applying hot and cold comprising a first end piece and a second end piece, said end pieces being disposed at one end of the device and being oriented at an angle of less than or equal to 90° with respect one another.

According to the method:
a hot temperature of between 35° C. and 48° C., preferably between 38° C. and 45° C., is applied to the application surface by the first end piece for a period of between 2 min and 6 min, for example equal to 5 min, for example equal to 3 min,
the application device is pivoted by at most a quarter turn, for a period of less than or equal to 5 seconds, preferably less than or equal to 2 seconds, or even less than or equal to 1 second, and
immediately upon pivoting a cold temperature of between 0° C. and 15° C., preferably between 4° C. and 12° C., preferably between 7° C. and 9° C., is applied to the application surface by the second end piece for a period of between 2 min and 6 min, for example equal to 4 min, for example equal to 3 min.

In general, the two end pieces are arranged so as to allow the passage from an application of hot to an application of cold and vice versa by way of a simple and rapid movement by the user, in this case a quarter turn of the device.

The temperature referred to as “hot” opens the pores of the epidermis and improves the penetration of the product into said pores, while the temperature referred to as “cold” closes the pores and improves the evenness of the surface condition of the epidermis.

The module for generating cold or hot may be, for example, a Peltier-effect module, by way of a heating resistor or by way of any other means capable of generating hot or cold.

The Peltier effect is known and will not be described in any more detail.

The end pieces for applying hot or cold are, for example, made of metal material, such as copper, stainless steel, aluminium or Zamak, or of ceramic, or of plastics material loaded with metal particles, or any other material capable of transferring heat energy.

Advantageously, prior to application of a temperature to the application surface, a sensitivity of the application surface between at least a first sensitivity referred to as “high” and a second sensitivity referred to as “low” is selected. It is possible to provide other types of sensitivity, for example an intermediate sensitivity, and a treatment mode chosen from a group comprising at least a first mode and a second mode is selected.

Advantageously, the selection of the sensitivity of the application surface is carried out manually by the user or automatically by an electronic control unit of the application device, said electronic control unit being configured to retrieve data originating from members for acquiring physical data of the user's epidermis and to analyse the condition of the application surface, for example the sensitivity or the condition of the pores of the user's epidermis.

For example, the period of application of hot and/or of cold is determined by the chosen treatment mode and/or by the electronic control unit as a function of the condition of the analysed application surface.

The period of application of hot may thus be adjusted as a function of the surface condition of the user's epidermis.

When the second sensitivity is selected, the temperature delivered by the first end piece is, for example, lower by 2° C. with respect to the temperature delivered when the first sensitivity is selected.

For example, when the second sensitivity is selected, the temperature delivered by the first end piece is between 35°° C. and 45° C.

When the second sensitivity is selected, the temperature delivered by the second end piece is, for example, greater by 2° C. with respect to the temperature delivered when the first sensitivity is selected.

For example, when the second sensitivity is selected, the temperature delivered by the second end piece is between 7° C. and 15° C.

For example, the first treatment mode is configured for treatment of the user's entire face and the second treatment mode is configured for treatment of certain precise areas of the face, such as the eye contour. In a variant, it is possible to provide other modes for treating the user's face.

For example, in the first treatment mode, the period of application of a hot temperature by the first end piece is equal to 3 min and the period of application of a cold temperature by the second end piece is equal to 4 min.

For example, in the second treatment mode, the period of application of a hot temperature by the first end piece is equal to 2 min and the period of application of a cold temperature by the second end piece is equal to 3 min.

Provision may also be made for an audible or visual signal to be emitted at the end of the step of applying heat in order to alert the user.

According to one embodiment, at least a first product is applied to the application surface prior to or at the same time as the step of applying a hot temperature and/or a cold temperature.

“Product” is understood to mean a cosmetic product, such as a liquid or semi-liquid product, such as a skincare product, such as a cream, a gel or a serum.

Thus, it is possible to combine the effects of temperature and of the cosmetic product on the user's epidermis.

Provision may also be made to apply a first product prior to or at the same time as the step of applying a hot temperature and a second product prior to or at the same time as the step of applying a cold temperature.

For example, the product is applied automatically by a product dispensing member disposed in the product application device.

Provision may also be made for the product dispensing member to be configured to dispense a first product via the first end piece and a second product via the second end piece.

For example, the hot temperature and the cold temperature are regulated by more or less 2° C. when the outside temperature is less than 24° C. and by more or less 5° C. when the outside temperature is greater than or equal to 24° C.

According to a second aspect, the invention relates to a device for alternately applying hot and cold to an application surface for the implementation of the application method as described above, comprising a first, hot end piece for the application of a hot temperature of between 35° C. and 48°° C., and a second, cold end piece for the application of a cold temperature of between 0° C. and 15° C., said end pieces being disposed at one end of the device and being oriented at an angle of less than or equal to 90° with respect to one another.

Advantageously, the application device comprises a plurality of members for acquiring physical data of the application surface, notably the user's epidermis, for example sensors, accelerometers, gyroscopes, contact sensors, ultrasound probe, a data retrieval module configured to retrieve the data originating from said members for acquiring physical data of the application surface, and an electronic control unit configured to analyse said application surface, notably in terms of thickness, sensitivity, appearance of the pores as a function of the physical data of the user's epidermis, and to automatically adjust the temperature and the period of application of a hot or cold temperature by the application end pieces.

The first end piece is configured to apply a hot temperature of between 35° C. and 48° C., preferably between 38° C. and 45° C., and the second end piece is configured to apply a cold temperature of between 0° C. and 15° C., preferably between 4° C. and 12° C., preferably between 7° C. and 9° C.

According to one embodiment, the application end pieces are connected to an external human-machine interface (HMI), for example on telephone, computer, etc., and/or to a diagnostic system.

For example, the application end pieces are connected to the human-machine interface wirelessly, for example via Bluetooth®, Wi-Fi or other wireless transmission means.

Advantageously, the application device comprises a member for packaging and dispensing product which is configured to dispense at least one dose of product to the outside of the application device.

According to one embodiment, the member for packaging and dispensing product is connected to each of the end pieces for applying hot and cold.

For example, the end pieces may for example be porous or comprise a perforated membrane for dispensing the product contained in the member for packaging and dispensing product.

In a variant, it is possible to provide an applicator that is independent of the end pieces, for example that is situated between said end pieces, in the form of a roll-on.

According to one embodiment, the application device comprises an actuator with movable plunger which is configured to displace the product contained in the member for packaging and applying product.

For example, the actuator is actuated manually by an activation member mounted on the outer surface of the device, or automatically by the electronic control unit.

In a variant, the product contained in the member for packaging and applying product is displaced by the quarter-turn movement of the device allowing the passage from one end piece to the other.

It is also possible to provide an assembly or kit for the implementation of the method as described above, comprising two application devices as described above, said hot and cold end pieces being arranged symmetrically between the right-hand and left-hand devices in order to treat two halves of the face at once.

Advantageously, the end pieces of the right-hand and left-hand devices are connected to one another.

The present invention will be better understood from studying the detailed description of embodiments that are given by way of entirely non-limiting example and are illustrated by the appended drawings, in which:

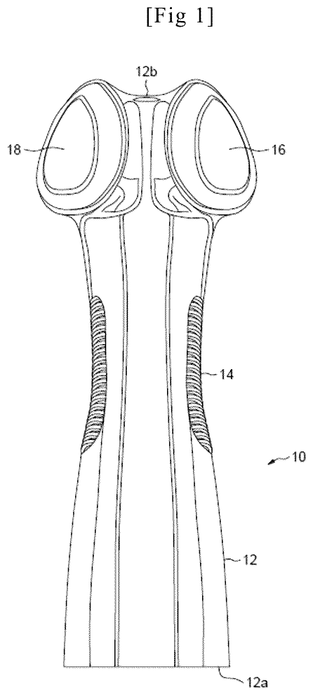

FIG. 1 is a perspective view of a device for alternately applying hot and cold for the implementation of a method for alternately applying hot and cold according to the invention;

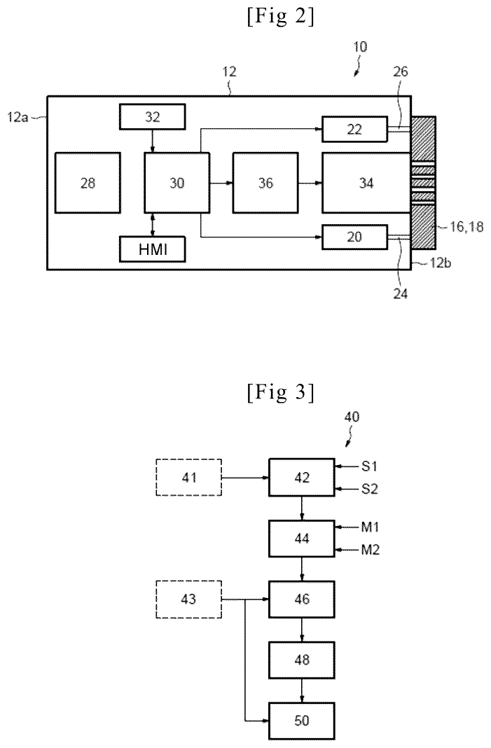

FIG. 2 is a schematic view in cross section of the application device in FIG. 1; and

FIG. 3 shows the steps of the method for alternately applying hot and cold according to the invention with the aid of the application device in FIG. 1.

A device 10 for alternately applying a hot and cold temperature to the epidermis of a user is illustrated in FIGS. 1 and 2.

The device 10 comprises a substantially elongate body 12 defining a gripping part 14 situated between a lower end 12a and an upper end 12b of the body 12.

The device 10 further comprises a first end piece 16 configured to apply a hot temperature and a second end piece 18 configured to apply a cold temperature.

Said end pieces 16, 18 are in this case disposed at the upper end 12b of the device 10 and are oriented at an angle of 90° with respect to one another. In general, the end pieces 16, 18 are oriented at an angle of less than or equal to 90° with respect one another.

The first end piece 16 is configured to generate a temperature of between 35° C. and 48° C., preferably between 38° C. and 45°° C. The temperature of the first end piece 16 for applying hot is advantageously constant, regardless of the outside temperature.

The second end piece 18 is configured to generate a temperature of between 0° C. and 15° C., preferably between 4° C. and 12° C., preferably between 7° C. and 9° C. The temperature of the second end piece 18 for applying cold is advantageously constant, regardless of the outside temperature.

The two application end pieces 16, 18 are disposed in two perpendicular planes allowing the passage from one end piece to the other by way of a quarter-turn movement of the device.

In general, the two end pieces 16, 18 are arranged so as to allow the passage from an application of a hot temperature to an application of a cold temperature and vice versa by way of a simple and rapid movement by the user, in this case a quarter turn of the device.

The two application end pieces 16, 18 are respectively connected to a hot generation device 20 or a cold generation device 22, each comprising a heat transfer body 24, 26 and a module for generating cold or hot (not shown) at the heat transfer body.

The module for generating cold or hot may be, for example, a Peltier-effect module, by way of a heating resistor or by way of any other means capable of generating hot or cold.

The Peltier effect is known and will not be described in any more detail.

The end pieces 16, 18 for applying hot or cold are, for example, made of metal material, such as copper, stainless steel, aluminium or Zamak, or of ceramic, or of plastics material loaded with metal particles, or any other material capable of transferring heat energy.

The temperature referred to as “hot” opens the pores of the epidermis and improves the penetration of the product into said pores, while the temperature referred to as “cold” closes the pores and improves the evenness of the surface condition of the epidermis.

The application device 10 further comprises an energy storage member 28, such as a battery, and an electronic control unit 30 configured to regulate the temperature transmitted to the application end pieces 16, 18.

The application device 10 comprises a plurality of members (not shown) for acquiring physical data of the user's epidermis, for example sensors, accelerometers, gyroscopes, contact sensors, ultrasound probe, and a data retrieval module 32 configured to retrieve the data originating from the members for acquiring physical data of the user's epidermis.

The electronic control unit 30 is thus configured to analyse the user's epidermis in terms of thickness, sensitivity, appearance of the pores as a function of the physical data of the user's epidermis and to automatically adjust the temperature and the period of application of hot or of cold by the application end pieces 16, 18.

The end pieces 16, 18 are, for example, connected to one another and/or to an external human-machine interface on telephone, computer, etc., and/or to a diagnostic system (not shown). For example, the application end pieces 16, 18 are connected to the human-machine interface wirelessly, for example via Bluetooth®, Wi-Fi or other wireless transmission means.

The application device 10 further comprises a member 34 for packaging and dispensing product which is mounted in the body 12.

“Product” is understood to mean a cosmetic product, such as a liquid or semi-liquid product, such as a skincare product, such as a cream, a gel or a serum.

The member 34 for packaging and dispensing product is configured to deliver a dose of product to the outside of the body 12 of the application device 10.

For example, the member 34 for packaging and dispensing product is connected to each of the end pieces 16, 18. In this case, the end pieces 16, 18 may for example be porous or comprise a perforated membrane for dispensing the product contained in the member 34 for packaging and dispensing product.

In a variant, it is possible to provide an applicator that is independent of the end pieces 16, 18, for example that is situated between said end pieces, in the form of a roll-on.

As illustrated, the product contained in the member 34 for packaging and applying product is displaced with the aid of an actuator 36 with movable plunger, which is actuated for example by an activation member (not shown) mounted on the outer surface of the body 12, or by the electronic control unit 30.

In a variant, the product contained in the member 34 for packaging and applying product is displaced by the quarter-turn movement of the device 10 allowing the passage from one end piece to the other.

The member 34 for packaging and applying productallows the cosmetic product to be dispensed during the application of hot and/or cold in order to combine the effects of temperature and of the cosmetic product on the user's epidermis.

As illustrated in FIG. 3, the method 40 for alternately applying hot and cold comprises a step 42 of selecting the sensitivity of the epidermis between a first sensitivity S1 referred to as “high” and a second sensitivity S2 referred to as “low”. It is possible to provide other types of sensitivity, for example an intermediate sensitivity.

The selection of the sensitivity in step 42 may be carried out manually by the user or automatically, in step 41, by the electronic control unit 30 subsequent to the acquisition of data originating from members (not shown) for acquiring physical data of the user's epidermis.

When the second sensitivity S2 is selected, the temperature of the first end piece 16 for applying hot delivers a temperature that is lower by 2° C. with respect to the temperature delivered when the first sensitivity S1 is selected.

When the second sensitivity S2 is selected, the temperature of the first end piece 16 delivers a temperature for example of between 35° C. and 45° C.

When the second sensitivity S2 is selected, the temperature of the second end piece 18 delivers a temperature that is greater by 2° C. with respect to the temperature delivered when the first sensitivity S1 is selected.

When the second sensitivity S2 is selected, the temperature of the second end piece 18 delivers a temperature of between 7° C. and 15° C.

The method 40 for alternately applying hot and cold further comprises a step 44 of selecting a treatment mode chosen from the group comprising at least a first mode M1 and a second mode M2.

The first treatment mode M1 is intended for treatment of the user's entire face and the second treatment mode M2 is intended for treatment of certain precise areas of the face, such as the eye contour.

In a variant, it is possible to provide other modes for treating the user's face.

The method 40 for alternately applying hot and cold further comprises a step 46 of applying heat by way of the first end piece 16 for a period of between 2 min and 6 min, for example equal to 5 min, for example equal to 3 min.

The period of application of hot is determined by the chosen treatment mode.

In an entirely non-limiting manner, the application method 40 comprises a step 43 of continuously analysing the user's epidermis during the step 46 of applying heat. The period of application of hot may thus be adjusted as a function of the surface condition of the user's epidermis.

At the end of the step 46 of applying heat, an audible or visual signal may be emitted in order to alert the user to the end of the step 46 of applying heat.

In step 48, the user pivots the application device 10 by at most a quarter turn, for a period of less than or equal to 5 seconds, preferably less than or equal to 2 seconds.

The method 40 for alternately applying hot and cold further comprises a step 50 of applying cold by way of the second end piece 18 for a period of between 2 min and 6 min, for example equal to 4 min, for example equal to 3 min.

The period of application of hot is determined by the chosen treatment mode and may be adjusted in the step 43 of analysing the user's epidermis.

At the end of the step 50 of applying cold, the method 40 ends.

For example, in the first treatment mode M1, the period of application of hot by the first end piece 16 is equal to 3 min and the period of application of cold by the second end piece 18 is equal to 4 min.

For example, in the second treatment mode M2, the period of application of a hot temperature by the first application end piece 16 is equal to 2 min and the period of application of a cold temperature by the second application end piece 18 is equal to 3 min.

In a variant, it is possible to provide a step (not shown) of applying the product prior to or at the same time as the step 50 of applying a cold temperature in the event of the use of a product dispensing member 34.

Provision may also be made to apply a first product prior to or at the same time as the step 48 of applying a hot temperature and a second product prior to or at the same time as the step 50 of applying a cold temperature. In this case, provision may be made for the product dispensing member 34 to be configured to dispense a first product via the first end piece 16 and a second product via the end piece 18.

It is also possible to provide a kit comprising two symmetrical application devices in order to simultaneously treat the two halves of the user's face.

Thus, it is possible to combine the effects of temperature and of the cosmetic product on the user's epidermis.

By virtue of the application method according to the invention, what is obtained, with a single application device, is the ability to alternate, in less than 5 seconds, the hot and cold contact on the user's epidermis. The device and the application method thus make it possible to treat the entire face by way of a simple and rapid movement.

1. Non-therapeutic method (40) for alternately applying hot and cold to an application surface, notably the epidermis of a user, with the aid of a device (10) for alternately applying hot and cold comprising a first end piece (16) extending in a first direction and a second end piece (18) extending in a second direction, said end pieces being disposed at one end of the device (10), the first direction and the second direction forming an angle of less than or equal to 90° with respect one another, wherein:
a hot temperature of between 35° C. and 48° C. is applied to the application surface by the first end piece (16) for a period of between 2 min and 6 min,
the application device (10) is pivoted by at most a quarter turn, for a period of less than or equal to 5 seconds, and
immediately upon pivoting a cold temperature of between 0° C. and 15° C. is applied to the application surface by the second end piece (18) for a period of between 2 min and 6 min, wherein prior to application of a temperature to the application surface, a sensitivity of the application surface between at least a first sensitivity (S1) and a second sensitivity (S2) is selected and a treatment mode chosen from a group comprising at least a first mode (M1) and a second mode (M2) is selected.

2. Method according to claim 1, wherein the selection of the sensitivity of the application surface is carried out manually by the user or automatically by an electronic control unit (30) of the application device (10), said electronic control unit being configured to retrieve data originating from members for acquiring physical data of the user's epidermis and to analyse the condition of the application surface.

3. Method according to claim 2, wherein the period of application of hot and/or of cold is determined by the chosen treatment mode and/or by the electronic control unit (30) as a function of the condition of the analysed application surface.

4. Method according to any one of the preceding claims, wherein when the second sensitivity (S2) is selected, the temperature delivered by the first end piece (16) for applying hot is lower by 2° C. with respect to the temperature delivered when the first sensitivity (S1) is selected.

5. Method according to any one of the preceding claims, wherein when the second sensitivity (S2) is selected, the temperature delivered by the second end piece (18) is greater by 2° C. with respect to the temperature delivered when the first sensitivity (S1) is selected.

6. Method according to any one of the preceding claims, wherein, at the end of the step of applying heat, an audible or visual signal is emitted to alert the user.

7. Method according to any one of the preceding claims, wherein at least a first product is applied to the application surface prior to or at the same time as the step of applying hot and/or cold.

8. Method according to claim 7, wherein the product is applied automatically by a product dispensing member (34) disposed in the product application device (10).

9. Method according to any one of the preceding claims, wherein the hot temperature and the cold temperature are regulated by more or less 2° C. when the outside temperature is less than 24° C. and by more or less 5°° C. when the outside temperature is greater than or equal to 24° C.

10. Device (10) for alternately applying hot and cold to an application surface for the implementation of the application method according to any one of the preceding claims, comprising a first, hot end piece (16) extending in a first direction for the application of a hot temperature of between 35° C. and 48° C., and a second, cold end piece (18) extending in a second direction for the application of a cold temperature of between 0° C. and 15° C., said end pieces being disposed at one end of the device (10), the first direction and the second direction forming an angle of less than or equal to 90° with respect to one another, said device (10) comprising a plurality of members for acquiring physical data of the application surface, a data retrieval module (32) configured to retrieve the data originating from said members for acquiring physical data of the application surface, and an electronic control unit (30) configured to analyse said application surface and to automatically adjust the temperature and the period of application of hot and/or of cold by the end pieces (16, 18).

11. Device (10) according to claim 10, further comprising a member (34) for packaging and dispensing product which is configured to deliver at least one dose of product to the outside of the application device (10).

12. Device (10) according to claim 11, wherein the member (34) for packaging and dispensing product is connected to each of the end pieces (16, 18).

13. Device (10) according to claim 12, comprising an actuator (36) with movable plunger which is configured to displace the product contained in the member (34) for packaging and applying product.

14. Assembly for the implementation of a method according to any one of claims 1 to 9, comprising a pair of devices according to any one of claims 10 to 13, the hot and cold end pieces being arranged symmetrically between the left-hand and right-hand devices.

15. Assembly according to claim 14, wherein the end pieces of the right-hand and left-hand devices are connected to one another and/or to an external human-machine interface and/or to a diagnostic system.
